(19) United States
(12) Patent Application Publication
(10) Pub. No.: US 2024/0415903 A1
(43) Pub. Date: Dec. 19, 2024

(54) GABA-PRODUCING GASTROINTESTINAL BACTERIA FOR SUPPORT OF MENTAL HEALTH

(71) Applicant: SYNBIOTIC HEALTH, INC., LINCOLN, NE (US)

(72) Inventors: Thomas Auchtung, Lincoln, NE (US); Mallory Van Haute, Gretna, NE (US); Katherine Chacon-Vargas, Lincoln, NE (US)

(21) Appl. No.: 18/745,165

(22) Filed: Jun. 17, 2024

Related U.S. Application Data
(60) Provisional application No. 63/508,330, filed on Jun. 15, 2023.

(51) Int. Cl. A61K 35/747; A61K 35/745; C12N 1/20; C12R 1/01; C12R 1/225; C12R 1/25

(52) U.S. Cl. CPC A61K 35/747; A61K 35/745; C12N 1/205; C12R 2001/01; C12R 2001/225; C12R 2001/25

(57) ABSTRACT

Provided are compositions and methods of using compositions including probiotics for treatment and prevention of a variety of pain disorders, neurological disorders, and/or physiological disorders. The compositions include one or more bacterium capable of producing γ-Aminobutyric acid (GABA). The bacterium is selected from Bifidobacterium adolescentis, Limosilactobacillus reuteri, and Lactiplantibacillus plantarum.

### RELATED APPLICATIONS

This application claims priority to Application Ser. No. 63/508,330, filed Jun. 15, 2023, and entitled “GABA-PRODUCING GASTROINTESTINAL BACTERIA FOR SUPPORT OF MENTAL HEALTH,” the entire contents of which are incorporated herein by reference.

### SEQUENCE LISTING XML

The instant application contains a sequence listing, which has been submitted in XML file format by electronic submission and is hereby incorporated by reference in its entirety. The XML file, created on Jun. 17, 2024 is named 93259.3.USU1_Sequence Listing.xml and is 38,774 bytes in size.

### TECHNICAL FIELD

This disclosure relates to compositions and methods relating to the improvement of various pathological complications via providing gamma-aminobutyric acid (GABA) produced in therapeutically effective amounts by GABA-producing bacterium.

### BACKGROUND

Psychobiotics are probiotics that can produce and deliver neuroactive substances such as gamma-aminobutyric acid (GABA) and serotonin (the “happy” chemical) that act on the brain-gut axis via reducing cortisol (the “stress” hormone) while increasing oxytocin (the “cuddle” hormone) levels (Selhub et al., 2014, Misra and Mohanty, 2019). In one example, γ-Aminobutyric acid (GABA) is a neurotransmitter that affects the mammalian nervous system by lessening the ability of a nerve cell to receive, create, and/or send chemical messages to other cells. GABA is known for its calming effects. In humans, various diseases have been associated with low levels of GABA, such as, for example, generalized anxiety, schizophrenia, major depressive disorder, seizures, and epilepsy. Altering GABA levels can also have an effect on a variety of other disorders such as, for example, sleep disorders, chronic pain, inflammation, and blood pressure.

GABA is produced by many organisms in nature, including animals, plants, and microbes. Because of the extensive biochemical signaling, GABA produced by bacteria in the gastrointestinal tract can affect many parts of the body, including the nervous system. In bacteria, the genes for synthesizing GABA are encoded by gadB (encoding a glutamate decarboxylase) and gadC (encoding a glutamate/gamma-aminobutyrate antiporter).

Bifidobacterium adolescentis is considered a psychobiotic and is a species in the phylum Actinobacteria that is common in the gastrointestinal tracts of primates (Lugli G A, et al. 2020. “Evolutionary development and co-phylogeny of primate-associated bifidobacteria.” Environ Microbiol 22:3375-3393), including humans, where it is abundant in the stool of both children and adults (Pasolli E, et al. 2017. “Accessible, curated metagenomic data through ExperimentHub.” Nat Methods 14:1023). Animal and in vitro studies suggest strains of B. adolescentis possess several beneficial activities, such as reducing anxiety and inflammation (Kawabata K, et al. 2018. “Functional properties of anti-inflammatory substances from quercetin-treated Bifidobacterium adolescentis.” Biosci Biotechnol Biochem 82:689-697, and Jang H M, et al. 2017. “Anxiolytic-like effect of Bifidobacterium adolescentis IM38 in mice with or without immobilisation stress.” Benef Microbes 9:123-132) and may protect against or improve recovery from several diseases (Long X, et al. 2021. “Bifidobacterium adolescentis alleviates liver steatosis and steatohepatitis by increasing fibroblast growth factor 21 sensitivity.” Front Endocrinol (Lausanne) 12:773340.) (Fan Z, et al. 2020. “Protective effects of Bifidobacterium adolescentis on collagen-induced arthritis in rats depend on timing of administration.” Food Funct 11:4499-4511). In addition, strains of B. adolescentis are of interest for their ability to metabolize prebiotics such as xylooligosaccharides, galactooligosaccharides, and arabinoxylan.

There remains a need in the art for improved methods and compositions of alleviating or preventing symptoms of various health disorders.

### BRIEF SUMMARY

Provided herein are methods and compositions for a probiotic strain selected by targeted in vivo (in the human gut) enrichment to aid with various health disorders.

In Example 1, a method of treating a mental health disorder in a subject in need thereof may include administering to the subject a composition comprising a therapeutically effective amount of a bacterium capable of producing γ-aminobutyric acid (GABA).

Example 2 relates to the method according to Example 1, wherein the bacterium is selected from Bifidobacterium adolescentis, Lactiplantibacillus plantarum, and Limosilactobacillus reuteri.

Example 3 relates to the method according to Example 1 or 2, wherein the bacterium is selected from Bifidobacterium adolescentis, Lactiplantibacillus plantarum, Limosilactobacillus reuteri, Bifidobacterium animalis, Bifidobacterium dentium, and Lactococcus lactis.

Example 4 relates to the method according to any one of Examples 1 to 3, wherein the bacterium is selected from Bifidobacterium adolescentis IVS-1, Limosilactobacillus reuteri K9-9, and Lactiplantibacillus plantarum IO-6.

Example 5 relates to the method according to any one of Examples 1 to 4, wherein the bacterium is Limosilactobacillus reuteri K9-9.

Example 6 relates to the method according to any one of Examples 1 to 5, wherein the mental health disorder includes one or more of: mood disorder, anxiety disorder, depression, attention deficit hyperactivity disorder (ADHD), social impairment, or panic disorders.

Example 7 relates to the method according to any one of Examples 1 to 6, wherein the administration of the composition increases GABA within a body of the subject.

Example 8 relates to the method according to any one of Examples 1 to 7, wherein the composition is administered by oral administration.

Example 9 relates to the method according to any one of Examples 1 to 8, wherein the bacterium is provided at a concentration of between about 10³ CFUs to about 10¹⁰ CFUs.

Example 10 relates to the method according to any one of Examples 1 to 9, wherein the composition further comprises a prebiotic comprising galactooligosaccharide (GOS), fructooligosaccharide (FOS), inulin, or a combination thereof.

Example 11 relates to the method according to any one of Examples 1 to 10, wherein the composition is further administered with a therapeutic agent comprising an antidepressant, an anxiolytic, stimulants, non-stimulants, or a combination thereof.

Example 12 relates to the method according to any one of Examples 1 to 11, wherein the therapeutic agent comprises a benzodiazepine, selective serotonin reuptake inhibitors (SSRIs), serotonin and norepinephrine reuptake inhibitors (SNRIs), tricyclic antidepressants (TCAs), monoamine oxidase inhibitors (MAOIs), methylphenidate, amphetamine, atomoxetine, viloxazine, guanfacine, clonidine, or a combination thereof.

Example 13 relates to the method according to any one of Examples 1 to 12, wherein the administration of the composition decreases a responsiveness of a nerve cell.

In Example 14, a of treating a disorder in a subject in need thereof may include administering to the subject a probiotic comprising a therapeutically effective amount of a bacterium capable of producing γ-aminobutyric acid (GABA), wherein the probiotic is administered at least one time.

Example 15 relates to the method according to Example 14, wherein the health disorders include one or more of: chronic pain, fatigue, blood pressure dysregulation, cardiovascular dysfunction, epilepsy, or inflammation.

Example 16 relates to the method according to Examples 14 or 15, wherein the bacterium is selected from Bifidobacterium adolescentis, Lactiplantibacillus plantarum, Limosilactobacillus reuteri, Bifidobacterium animalis, Bifidobacterium dentium, and Lactococcus lactis.

Example 17 relates to the method according to any one of Examples 14 to 16, wherein the bacterium is Bifidobacterium adolescentis IVS-1.

Example 18 relates to the method according to any one of Examples 14 to 17, wherein the bacterium is Limosilactobacillus reuteri K9-9.

Example 19 relates to the method according to any one of Examples 14 to 18, wherein the at least one time comprises one time daily.

In Example 20, a method of treating a mental health disorder in a subject in need thereof may include administering to the subject a composition comprising a therapeutically effective amount of a bacterium capable of producing γ-aminobutyric acid (GABA), wherein the composition comprises a probiotic, the probiotic provided at a concentration of between about 10³ CFUs to about 10¹⁰ CFUs, and wherein the bacterium is selected from Bifidobacterium adolescentis, Lactiplantibacillus plantarum, and Limosilactobacillus reuteri.

While multiple embodiments are disclosed, still other embodiments of the disclosure will become apparent to those skilled in the art from the following detailed description, which shows and describes illustrative embodiments of the disclosure. As will be realized, the disclosure is capable of modifications in various obvious aspects, all without departing from the spirit and scope of the disclosure. Accordingly, the drawings and detailed description are to be regarded as illustrative in nature and not restrictive.

### BRIEF DESCRIPTION OF THE DRAWINGS

In the accompanying figures, like elements are identified by like reference numerals among the several preferred embodiments of the present disclosure.

FIG. 1 is a genome map of B. adolescentis IVS-1.

FIG. 2 is a graph showing the amount of GABA produced by B. adolescentis IVS-1, L. plantarum IO-6, and L. reuteri K9-9 in comparison to several other probiotic strains when grown in a low pH MRS media plus glutamate.

### DETAILED DESCRIPTION

The foregoing and other features and advantages of the disclosure are apparent from the following detailed description of exemplary embodiments, read in conjunction with the accompanying drawings. The detailed description and drawings are merely illustrative of the disclosure rather than limiting, the scope of the disclosure being defined by the appended claims and equivalents thereof.

Embodiments of the disclosure will now be described with reference to the Figures, wherein like numerals reflect like elements throughout. The terminology used in the description presented herein is not intended to be interpreted in any limited or restrictive way, simply because it is being utilized in conjunction with detailed description of certain specific embodiments of the disclosure. Furthermore, embodiments of the disclosure may include several novel features, no single one of which is solely responsible for its desirable attributes, or which is essential to practicing the disclosure described herein.

The use of the terms “a” and “an” and “the” and similar referents in the context of describing the disclosure are to be construed to cover both the singular and the plural, unless otherwise indicated herein or clearly contradicted by context. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

Recitation of ranges of values herein are merely intended to serve as a shorthand method of referring individually to each separate value falling within the range, unless otherwise indicated herein, and each separate value is incorporated into the specification as if it were individually recited herein. The word “about,” when accompanying a numerical value, is to be construed as indicating a deviation of up to and inclusive of about one log from the stated numerical value. For example, the term “about” may be construed to indicate a deviation of between 0.1 to 10 times the stated value. The use of any and all examples, or exemplary language (“e.g.” or “such as”) provided herein, is intended merely to better illuminate the disclosure and does not pose a limitation on the scope of the disclosure unless otherwise claimed. No language in the specification should be construed as indicating any nonclaimed element as essential to the practice of the disclosure.

References to “one embodiment,” “an embodiment,” “example embodiment,” “various embodiments,” etc., may indicate that the embodiment(s) of the disclosure so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

As used herein, “isolated” refers to the obtaining of a population of microbial cells in which at least about 80% (e.g., about 85%, 90%, 95%, 99% or 100%) of the cells are of a particular strain, such as the Bifidobacterium adolescentis, the Lactiplantibacillus plantarum, and Limosilactobacillus reuteri strains described herein.

The expression “probiotics” is referred to herein as a composition which comprises probiotic microorganisms. Probiotic bacteria are defined as live bacteria, which when administered in adequate amounts confer a health benefit on the host. Probiotic microorganisms have been defined as “Live microorganisms which when administered in adequate amounts confer a health benefit on the host” (FAO/WHO 2002).

The expression “psychobiotics” is recently created to designate an emerging class of probiotics with psychiatric applications (Dinan et al., 2013). As referred to herein, psychobiotics are probiotics that can produce and deliver neuroactive substances such as gamma-aminobutyric acid (GABA) and serotonin (the “happy” chemical) that act on the brain-gut axis via reducing cortisol (the “stress” hormone) while increasing oxytocin (the “cuddle” hormone) levels (Selhub et al., 2014, Misra and Mohanty, 2019).

The expression “prebiotic” is referred to a composition or a component of a composition which is selectively utilized by host microorganisms conferring a health benefit”. Prebiotics are generally non-viable food components that are specifically fermented in the colon by bacteria thought to be of positive value, e.g., bifidobacteria, lactobacilli, and other short-chain fatty acid producing microorganisms. Prebiotics are also known as an ingredient that allows specific changes, both in the composition and/or activity in the gastrointestinal microbiota that confers benefits upon host well-being and health. The combined administration of a probiotic strain with one or more prebiotic compounds, when designed optimally, may enhance the growth of the administered probiotic in vivo resulting in additional or more pronounced health benefits, and is termed a synbiotic. A synbiotic is formally defined as “a mixture, comprising live microorganisms and substrate(s) selectively utilized by host microorganisms, that confers a health benefit on the host”. Well characterized prebiotics include, for example, galactooligosaccharide (GOS), fructooligosaccharide (FOS), and inulin. GOS and FOS refer to a group of oligomeric, non-digestible carbohydrates that are often produced from monomers using glycosidases to catalyze transgalactosylation reactions. These carbohydrates are often recalcitrant to digestion by host-secreted enzymes in the small intestine, such that they reach the colon intact and are available to the colonic microbiota. It would be understood by those skilled in the art that other compounds that fall within the definition of a prebiotic also can be used in the methods described herein.

The expression “postbiotic” refers to inanimate microorganisms and/or their components that confer health benefits on the host. (International Scientific Association for Probiotics and Prebiotics (ISAPP), 2021). For example, a postbiotic may be microorganisms that are no longer living but may include intact cells or other structural fragments, such as cell walls. Postbiotics may also comprise substances or byproducts of microorganisms, such as metabolites, proteins, or peptides, which may contribute to the overall health effect conferred by a postbiotic.

As used herein, a “subject” can refer to a human or a non-human. Representative non-human subjects include, without limitation, livestock (e.g., swine, cow, horse, goat, and sheep), poultry (e.g., fowls such as chicken and turkey), and companion animals (e.g., pets such as dogs and cats).

As used herein, “a subject in need thereof” means a human or non-human mammal that exhibits one or more symptoms or indications of a variety of health disorders, such as pain or nervous system disorders. In some embodiments, the pain disorders may include acute, chronic, or inflammatory pain. In some embodiments, nervous system disorders may include anxiety disorder, depression, dissociative disorder, personality disorder, cognitive disorder, mood disorder, affective disorder, neurodegenerative disorder, convulsive disorder, Parkinson's disease, Alzheimer's disease, epilepsy, schizophrenia, psychosis, Huntington's disease, Gilles de la Tourette syndrome, fainting, hypoxia, brain disorders, neurodegenerative disorders, panic, insomnia, addictive disorders, or restless leg syndrome. In many embodiments, the term “subject” may be interchangeably used with the term “patient”.

In a further embodiment, the composition further comprises a pharmaceutically acceptable carrier. As used herein, the term “pharmaceutically acceptable carrier” means one or more solid or liquid filler diluents or encapsulating substances which are suitable for administration to a human or an animal and which is/are compatible with the active probiotic organisms. The term “compatible” relates to components of the pharmaceutical composition which are capable of being comingled with the Bifidobacterium adolescentis strain IVS-1, Lactiplantibacillus plantarum IO-6, and Limosilactobacillus reuteri K9-9, further described herein, or a mutant strain thereof in a manner enabling no interaction that would substantially reduce the probiotic efficacy of the organisms selected for the present disclosure under ordinary use conditions. Pharmaceutically acceptable carriers must be of a sufficiently high purity and a sufficiently low toxicity to render them suitable for administration to humans and animals being treated.

A bacterial “strain” as used herein refers to a bacterium which remains genetically unchanged when grown or multiplied. The multiplicity of identical bacteria is included. “Wild type strain” refers to the non-mutated form of a bacterium, as found in nature. In the present context, the term “derived strain” should be understood as a strain derived from a mother strain by means of e.g., genetic engineering, normal laboratory and commercial production culturing, radiation and/or chemical treatment, and/or selection, adaptation, screening, etc. In specific embodiments the derived strain is a functionally equivalent mutant, e.g., a mutant that has substantially the same, or improved, properties (e.g., regarding probiotic properties) as the mother strain. Such a derived strain is a part of the present disclosure. The term “derived strain” includes a strain obtained by subjecting a strain of the present disclosure to any conventionally used mutagenization treatment including treatment with a chemical mutagen such as ethane methane sulphonate (EMS) or N-methyl-N′-nitro-N-nitroguanidine (NTG), UV light, or to a spontaneously occurring mutant.

A “mutant bacterium” or a “mutant strain” refers to a natural (spontaneous, naturally occurring) mutant bacterium or an induced mutant bacterium comprising one or more mutations in its genome (DNA) which are absent in the wild type DNA. An “induced mutant” is a bacterium where the mutation was induced by human treatment, such as treatment with any conventionally used mutagenization treatment including treatment with chemical mutagens, such as a chemical mutagen comprising (i) a mutagen that associates with or become incorporated into DNA such as a base analogue, e.g., 2-aminopurine or an interchelating agent such as ICR-191, or (ii) a mutagen that reacts with the DNA including alkylating agents such as nitrosoguanidine or hydroxylamine, or ethane methyl sulphonate (EMS) or N-methyl-N′-nitro-N-nitroguanidine (NTG), UV- or gamma radiation etc. In contrast, a “spontaneous mutant” or “naturally occurring mutant” has not been mutagenized by man. A derived strain, such as a mutant, may have been subjected to several mutagenization treatments (a single treatment should be understood one mutagenization step followed by a screening/selection step), but typically no more than 20, no more than 10, or no more than 5, treatments are carried out. In specific embodiments of derived strains, such as mutants, less than 1%, less than 0.1%, less than 0.01%, less than 0.001% or even less than 0.0001% of the nucleotides in the bacterial genome have been changed (such as by replacement, insertion, deletion, or a combination thereof) compared to the mother strain. Mutant bacteria as described above are non-GMO, i.e., not modified by recombinant DNA technology. As an alternative to the above preferred method of providing the mutant by random mutagenesis, it is also possible to provide such a mutant by site-directed mutagenesis, e.g., by using appropriately designed PCR techniques or by using a transposable element which is integrable in bacterial replicons. When the mutant is provided as a spontaneously occurring mutant the above wild-type strain is subjected to the selection step without any preceding mutagenization treatment.

### Compositions

The present disclosure provides for compositions and methods of utilizing probiotics for preventing or treating disorders associated with low levels of GABA in a subject. In some aspects, the disorder may include mental health disorders. In further aspects, the compositions and methods of the disclosure may be used to prevent or treat symptoms of disorders associated with low levels of GABA.

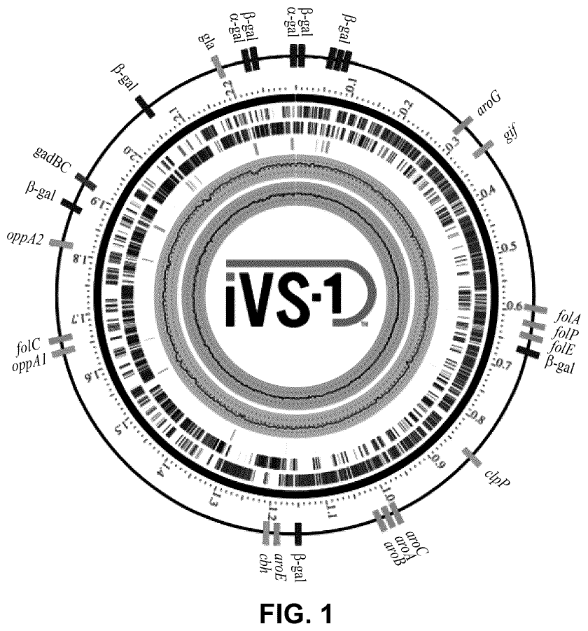

In some embodiments, the composition includes a probiotic. The probiotic may comprise Bifidobacterium adolescentis (B. adolescentis). In some embodiments, the probiotic comprises Bifidobacterium adolescentis strain IVS-1. FIG. 1 shows a genome map of B. adolescentis IVS-1. Bifidobacterium adolescentis strain IVS-1 is an ecologically-adapted probiotic strain with the genetic capacity to be competitive for lactose and lactose-like carbohydrates in the human gut. Bifidobacterium adolescentis strain IVS-1 was identified by the process of in vivo selection (IVS) after being enriched in the human gut through consumption of the probiotic GOS (which is chemically similar to lactose) by an individual who naturally hosted the strain. Therefore, the Bifidobacterium adolescentis strain IVS-1 disclosed herein are provided at a concentration level much higher than would be found naturally within an individual subject.

Preferably, the present composition contains a Bifidobacterium adolescentis strain IVS-1 which has at least 95% identity with the 16S rRNA gene sequence when compared to the type strain identified in SEQ ID NO:1 below. In embodiments, the present composition contains Bifidobacterium adolescentis strain IVS-1 which has at least 97% identity, at least 98% identity, at least 98.5% identity, at least 99% identity, at least 99.5% identity, or at least 99.9% identity with the 16S rRNA gene sequence when compared to the type strain identified in SEQ ID NO: 1 below, which is further provided within a sequence listing in XML format, incorporated herein by reference.

Sequence of 16S rRNA gene from microbial
strain Bifidobacterium adolescentis strain
IVS-1
(SEQ ID NO: 1)
TGCAGTCGAA CGGGATCCCA GGAGCTTGCT CCTGGGTGAG
AGTGGCGAAC GGGTGAGTAA TGCGTGACCG ACCTGCCCCA
TACACCGGAA TAGCTCCTGG AAACGGGTGG TAATGCCGGA
TGCTCCAGTT GACCGCATGG TCCTCTGGGA AAGCTTTTGC
GGTATGGGAT GGGGTCGCGT CCTATCAGCT TGATGGCGGG
GTAACGGCCC ACCATGGCTT CGACGGGTAG CCGGCCTGAG
AGGGCGACCG GCCACATTGG GACTGAGATA CGGCCCAGAC
TCCTACGGGA GGCAGCAGTG GGGAATATTG CACAATGGGC
GCAAGCCTGA TGCAGCGACG CCGCGTGCGG GATGACGGCC
TTCGGGTTGT AAACCGCTCT TGACTGGGAG CAAGCCCTTC
GGGGTGAGTG TACCTTTCGA ATAAGCACCG GCTAACTACG
TGCCAGCAGC CGCGGTAATA CGTAGGGTGC AAGCGTTATC
CGGAATTATT GGGCGTAAAG GGCTCGTAGG CGGTTCGTCG
CGTCCGGTGT GAAAGTCCAT CGCTTAACGG TGGATCCGCG
CCGGGTACGG GCGGGCTTGA GTGCGGTAGG GGAGACTGGA
ATTCCCGGTG TAACGGTGGA ATGTGTAGAT ATCGGGAAGA
ACACCAATGG CGAAGGCAGG TCTCTGGGCC GTCACTGACG
CTGAGGAGCG AAAGCGTGGG GAGCGAACAG GATTAGATAC
CCTGGTAGTC CACGCCGTAA ACGGTGGATG CTGGATGTGG
GGACCATTCC ACGGTCTCCG TGTCGGAGCC AACGCGTTAA
GCATCCCGCC TGGGGAGTAC GGCCGCAAGG CTAAAACTCA
AAGAAATTGA CGGGGGCCCG CACAAGCGGC GGAGCATGCG
GATTAATTCG ATGCAACGCG AAGAACCTTA CCTGGGCTTG
ACATGTTCCC GACAGCCGTA GAGATACGGT CTCCCTTCGG
GGCGGGTTCA CAGGTGGTGC ATGGTCGTCG TCAGCTCGTG
TCGTGAGATG TTGGGTTAAG TCCCGCAACG AGCGCAACCC
TCGCCCTGTG TTGCCAGCAC GTCGTGGTGG GAACTCACGG
GGGACCGCCG GGGTCAACTC GGAGGAAGGT GGGGATGACG
TCAGATCATC ATGCCCCTTA CGTCCAGGGC TTCACGCATG
CTACAATGGC CGGTACAACG GGATGCGACA CTGTGAGGTG
GAGCGGATCC CTTAAAACCG GTCTCAGTTC GGATTGGAGT
CTGCAACCCG ACTCCATGAA GGCGGAGTCG CTAGTAATCG
CGGATCAG

In some aspects, measured by 16S rRNA gene sequencing. B. adolescentis (such as strain IVS-1) was identified as a taxa of interest that appeared to increase upon lactose feeding, as shown in Table 1. Operational taxonomic units (OTUs) constructs “mathematically” defined taxa, which is widely accepted and applied to describe bacterial communities using amplicon sequencing of 16S rRNA gene.

In some embodiments, the probiotic may comprise Lactiplantibacillus plantarum (L. plantarum). In some embodiments, the probiotic comprises Lactiplantibacillus plantarum strain IO-6. L. plantarum are homofermentative, aerotolerant Gram-positive bacteria. L. plantarum has one of the largest genomes known among the lactic acid bacteria and is a very flexible and versatile species. It is estimated to grow between pH 3.4 and 8.8. Lactiplantibacillus plantarum can grow in the temperature range 12° C. to 40° C. For example, Lactiplantibacillus plantarum may grow at 15° C. (59° F.), but not at 45° C. (113° F.). L. plantarum may produce both isomers of lactic acid (D and L). Many lactobacilli including L. plantarum are unusual in that they can respire oxygen and express cytochromes if heme and menaquinone are present in the growth medium.

Preferably, the present composition contains a Lactiplantibacillus plantarum strain IO-6, which has at least 78% identity with the 16S rRNA gene sequence when compared to the type strain identified in SEQ ID NO:1. In some embodiments, the present composition contains Lactiplantibacillus plantarum strain IO-6 which has at least 78.5% identity, at least 79% identity, at least 79.5% identity, at least 80% identity, at least 80.5% identity, or at least 81% identity, with the 16S rRNA when compared to the type strain identified in SEQ ID NO:1, which is further provided within a sequence listing in XML format.

In some embodiments, the probiotic may comprise Limosilactobacillus reuteri (L. reuteri). In some embodiments, the probiotic comprises Limosilactobacillus reuteri strain K9-9. L. reuteri is a lactic acid bacterium in the phylum Firmicutes. Limosilactobacillus reuteri is considered a psychobiotic. L. reuteri is a well-studied probiotic bacterium that exerts beneficial health effects due to several metabolic mechanisms that enhance the production of anti-inflammatory cytokines and modulate the gut microbiota by the production of antimicrobial molecules, including reuterin. In some examples, the optimum temperature range for lactobacilli growth may be between 30-40° C. and the optimum pH conditions for lactobacilli growth may be a pH between 5.5-6.2. However, the Lactobacillus genus is diversified, and certain bacteria can grow in temperatures ranging from 2 to 53° C. and PH levels varying between 4.5 and 6.5. Lactobacilli's main antibacterial activity is due to the release of lactic acid, which lowers the pH of the surrounding environment and the internal cell pH of pathogens. However, lactic acid is not the only organic acid involved in the antibacterial activity. Lactobacilli can produce other organic acids, such as acetic, propionic and phenyl lactic acids, which contribute both to the drop in pH and the potential inhibition of the growth of pathogenic microorganisms. Moreover, lactobacilli can produce a wide variety of antimicrobial molecules, including low-molecular-mass compounds such as hydrogen peroxide, carbon dioxide, ethanol, diacetyl, and acetaldehyde, as well as more complex molecules like bacteriocins, reuterin and reutericyclin, which are the final products of metabolism performed by Limosilactobacillus reuteri strains.

Preferably, the present composition contains a Limosilactobacillus reuteri strain K9-9, which has at least 79% identity with the 16S rRNA gene when compared to the type strain identified in SEQ ID NO:1. In embodiments, the present composition contains Limosilactobacillus reuteri strain K9-9 which has at least 79.5% identity, at least 80% identity, at least 80.5% identity, at least 81% identity, at least 81.5% identity, or at least 82% identity, with the 16S rRNA when compared to the type strain identified in SEQ ID NO: 1, which is further provided within a sequence listing in XML format.

Although it appears GABA is not produced by most bacterial species, some strains of species, including Bifidobacterium adolescentis, Lactiplantibacillus plantarum, Limosilactobacillus reuteri, Bifidobacterium animalis, Bifidobacterium dentium, and Lactococcus lactis, appear to encode one or more of these genes and produce measurable amounts of GABA. The amount of GABA that is produced varies under different conditions, and from strain to strain.

When analyzing the genome of a human-isolated strain Bifidobacterium adolescentis IVS-1, a human-isolated strain Lactiplantibacillus plantarum IO-6, and a dog-isolated strain Limosilactobacillus reuteri K9-9, it was discovered that one or more of the gad genes (e.g., gadA, gadB, gadC) were present and had a high identity to the gad genes of other known GABA-producing strains.

GABA (gamma-aminobutyric acid) is an amino acid that functions as the primary inhibitory neurotransmitter for the central nervous system (CNS). GABA is synthesized in the cytoplasm of the presynaptic neuron from the precursor glutamate by the enzyme glutamate decarboxylase, an enzyme which uses vitamin B6 (pyridoxine) as a cofactor. The action of GABA in the central nervous system is carried out by its interaction with specific GABAergic receptors (divided into GABA-A and GABA-B receptors), leading to inhibition of the nerve impulse. Outside the nervous system, the GABAergic system has been described in various tissues and organs of the human body (intestines, stomach, pancreas, kidneys, lungs, liver, and others).

GABA works at inhibitory synapses in the brain by binding to specific transmembrane receptors in the plasma membrane, both in presynaptic and post-synaptic neuronal processes. This binding causes the ion channel to open, allowing either negatively charged chloride ions to enter the cell or positively charged potassium ions to flow out of the cell. This effect results in a negative change in transmembrane potential, usually resulting in hyperpolarization. Three common GABA receptors, GABA A, GABA B, and GABA C have been identified. GABA A and GABA C are ionotropic receptors, while GABA B is a G protein-coupled metabotropic receptor. Low GABA levels are associated with many diseases, such as those listed herein.

The instant disclosure includes methods for treatment and/or prevention of various health disorders that may be associated with low levels of GABA. In particular, mental health disorders. As provided herein, a method for treating or preventing health disorders, such as mental health disorders, includes administration of an effective amount of the disclosed compositions to a subject in need thereof.

According to certain embodiments, the subject in need thereof has been diagnosed with a pain disorder. In some embodiments, the subject has been diagnosed with a physiological disorder. In exemplary embodiments, the subject has been diagnosed with a nervous system disorder, e.g., a mental health disorder.

For example, in some embodiments, the pain disorders may include acute, chronic, or inflammatory pain. In some embodiments, the physiological disorders may include fatigue, blood pressure dysregulation, cardiovascular dysfunction, or inflammation. In some embodiments, nervous system disorders may include anxiety disorder, depression, dissociative disorder, personality disorder, cognitive disorder, mood disorder, affective disorder, attention deficit hyperactivity disorder (ADHD), social impairment, convulsive disorder, Parkinson's disease, Alzheimer's disease, epilepsy, schizophrenia, psychosis, Huntington's disease, Gilles de la Tourette syndrome, fainting, hypoxia, brain disorders, neurodegenerative disorders, panic, insomnia, addictive disorders, or restless leg syndrome.

The composition may include mixed cultures of live microorganisms rather than single strains. For example, the composition may include one or more bacterial strains (e.g., Bifidobacterium adolescentis IVS-1, Lactiplantibacillus plantarum 10-6, Limosilactobacillus reuteri K9-9, etc.), and one or more yeast strains. In some embodiments, the composition may include one bacterium (e.g., Bifidobacterium adolescentis IVS-1, or Lactiplantibacillus plantarum 10-6, or Limosilactobacillus reuteri K9-9, etc.). In some embodiments, the composition may further comprise a prebiotic comprising: galactooligosaccharide (GOS), fructooligosaccharide (FOS), inulin, or a combination thereof. The prebiotic may serve to help the one or more bacterial strains remain in the gastrointestinal tract of the subject for a longer period of time. However, this is not required. The dosage of the composition administered will be dependent upon the age, health, and weight of the recipient, kind of concurrent treatment, if any, frequency of treatment, and the nature of the effect desired. In some embodiments, the composition may comprise a postbiotic comprising one or more byproducts (e.g., GABA) of the bacterial strains (e.g., Bifidobacterium adolescentis IVS-1, or Lactiplantibacillus plantarum IO-6, or Limosilactobacillus reuteri K9-9). In some embodiments, the dose, or the therapeutically effective amount, of the composition (e.g., probiotic) of the disclosure is from about 10³ CFUs to about 10¹⁰ CFUs. In some embodiments, the dose may contain up to 50 billion CFUs. In some embodiments, the composition may be administered at least one time. In some embodiments, at least one time may comprise one time daily. In other embodiments, the composition may be administered twice daily, three times daily, once weekly, twice weekly, once monthly, twice monthly, or any other time frame as desired and/or prescribed.

According to certain embodiments, the composition may be administered orally. For example, the composition may in a pill form, a powdered form, or a liquid form, and may be intended to be swallowed by the subject. In other embodiments, the composition may be intended to be a sublingual or a buccal composition. In some embodiments, the composition (e.g., probiotic) may be incorporated into a food source such as, but not limited to, yogurt, animal feed, breads, milk products, etc.

In some embodiments, the composition, e.g., a probiotic, when administered at regular intervals as prescribed, may increase GABA within the subject. The increase of GABA may then decrease a responsiveness of a nerve cell. This may treat a disorder (as listed herein) and/or prevent a disorder. In some embodiments, the composition may be administered with a therapeutic agent comprising one or more of: an antidepressant, an anxiolytic, a stimulant, and a non-stimulant. For example, the therapeutic agent may comprise a benzodiazepine, selective serotonin reuptake inhibitors (SSRIs), serotonin and norepinephrine reuptake inhibitors (SNRIs), tricyclic antidepressants (TCAs), monoamine oxidase inhibitors (MAOIs), methylphenidate, amphetamine, atomoxetine, viloxazine, guanfacine, clonidine, or a combination thereof. In such cases, the combination of the composition and the therapeutic agent may treat a disorder (as listed herein) and/or prevent a disorder.

### EXAMPLES

The following examples are put forth so as to provide those of ordinary skill in the art with a complete disclosure and description of certain examples of how the compounds, compositions, articles, devices and/or methods claimed herein are made and evaluated and are intended to be purely exemplary of the disclosure and are not intended to limit the scope of what the inventors regard as their disclosure. However, those of skill in the art should, in light of the present disclosure, appreciate that many changes can be made in the specific embodiments which are disclosed and still obtain a like or similar result without departing from the spirit and scope of the disclosure.

### Example 1

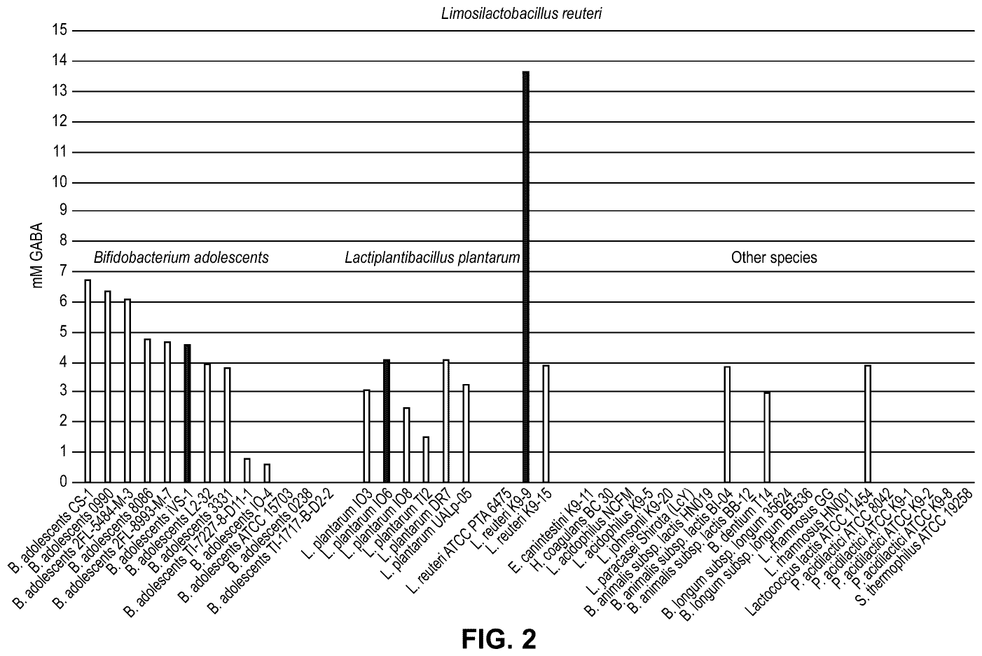

In one example, cells of B. adolescentis IVS-1, L. plantarum IO-6, and L. reuteri K9-9 were grown for 48 hr in MRS (Difco) media supplemented with the GABA precursor glutamate and adjusted to pH 5.6. Cultures were centrifuged and the supernatants collected. Supernatants were frozen then freeze-dried for 48 hr. A small, equivalent aliquot of the material was added to methanol and chloroform for extraction of amino acids. These samples were dried down, dissolved in 20 mM HCl, and derivatized using the AccQ-Tag reagent from Waters. An HPLC method for amino acids detection and quantification was run using the kit mobile phase A and B. The elution conditions are described in the Waters Free Amino Acids UPLC manual. In addition to the test samples, a standard curve with known concentrations of GABA was also analyzed. The concentration of GABA in the samples was calculated from the raw data using an equation generated from the standard curve. B. adolescentis IVS-1, L. plantarum IO-6, and L. reuteri K9-9 all produced significant amounts of GABA, as shown in FIG. 2.

As shown in FIG. 2, additional bacterium produced significant amounts of GABA. For example, in some aspects, other GABA-producing strains suitable for use to improve mental health may include, but are not limited to, B. adolescentis, L. plantarum, L. reuteri, and B. dentium. In further examples, Bifidobacterium animalis subsp. lactis B1-04, Bifidobacterium dentium T14, and Lactococcus lactis ATCC 11454 are shown to produce significant amounts of GABA.

### Example 2

In another example, the genome of the human-isolated strain Bifidobacterium adolescentis IVS-1, human-isolated strain Lactiplantibacillus plantarum IO-6, and dog-isolated strain Limosilactobacillus reuteri K9-9 were analyzed. It was discovered that one or more of the gad genes were present and had high identity to the gad genes of other known GABA-producing strains. The production of GABA was examined in these strains in comparison to other strains.

B. adolescentis IVS-1 was isolated from the fecal sample of a human that consumed increasing amounts of GOS. It has been shown to clinically improve intestinal barrier integrity and persist in several subjects four weeks after treatment. A draft genome was previously sequenced by Roche 454 sequencing and assembled into 145 contigs (NCBI Assembly ASM82986v1). A genome map of B. adolescentis IVS-1 is shown in FIG. 1.

Cells were grown to late exponential phase in 300 ml Reinforced Clostridial Medium (RCM; BD Difco). The culture was centrifuged, and the pellet sent to CD Genomics (Shirley, NY, USA). Total DNA was extracted using a DNeasy UltraClean Microbial Kit (Qiagen), sheared to 10 Kb using a g-TUBE (Covaris), and constructed into a library using a SMRTbell kit. A total of 416,354 reads were sequenced on a PacBio Sequel II, with an average length of 9,546 bp (N50=10,333 bp), mean depth of 1,473, and 100% coverage. Genome assembly into a single scaffold was performed with Flye (v2.8.3-b1695) and Canu (v1.6) software using default parameters.

The B. adolescentis IVS-1 genome was discovered to have 2,306,390 bp, with a GC content of 59.62%. This is similar to other previously sequenced B. adolescentis strains (2.09-2.40 Mbp and 59.2-59.9%, respectively). Using PATRIC, IVS-1 proteins homologous to those in the PGFam database were identified, aligned with MUSCLE, and analyzed with RaxML, confirming IVS-1 clusters with other B. adolescentis.

Genome annotation with RAST showed a total of 1,885 protein coding genes, 13 rRNA, 56 tRNA, and 1 CRISPR region. Prediction of gene functions were based on Gene Ontology (GO), Kyoto Encyclopedia of Genes and Genomes (KEGG), Clusters of Orthologous Groups (COG) and Swiss-Prot. A total of 1,317 predicted proteins had a functional assignment, 516 had an EC number, 444 had GO assignments, and 385 were associated with molecular pathways. Based on subsystem analysis most genes were associated with metabolism (350 genes), followed by protein processing (183 genes), stress response (64 genes) and DNA processing (72 genes).

Additional analyses were performed to examine genes that potentially confer benefits to the host. Functional annotation included CAZy profiling using dbCAN3 (18) with HMMER (v3.1b2). Using the parameters of E-Value≤le-15 and coverage≤0.35, a total of 102 enzyme families were found, belonging to four categories. There were 56 glycoside hydrolases (GH), 26 carbohydrate-binding modules, 18 glycosyl transferases, and 2 carbohydrate esterases. The most abundant enzymes were GH13, involved in complex glycans degradation, GH3, GH43, and GH51, involved in degradation of plant-derived carbohydrates, and GH42, related to the ability to grow on galactose-containing compounds, such as GOS and lactose. The genome of IVS-1 has two α-galactosidase genes, relevant for the metabolism of carbohydrates such as raffinose. In addition, IVS-1 was discovered to have nine different β-galactosidases.

The IVS-1 genome was examined for genes that may be important for probiotic activity, including survival and persistence in the gut, production of the vitamin folate, and the synthesis of the inhibitory neurotransmitter gamma-aminobutyric acid (GABA). The IVS-1 genome was determined to encode cbh, clpP, gla, glf, and oppA, which may be important for resisting bile, osmotic, and/or acid stress. In addition, IVS-1 was determined to possess fol genes for de novo folate production, as well as aro and pab genes for synthesis of the folate precursors chorismate and para-aminobenzoic acid. In some aspects, other GABA-producing strains suitable for use to improve mental health may include, but are not limited to, B. adolescentis, L. plantarum, L. reuteri, and B. dentium, as shown in FIG. 2.

What is claimed is:

1. A method of treating a mental health disorder in a subject in need thereof, the method comprising:
administering to the subject a composition comprising a therapeutically effective amount of a bacterium capable of producing γ-aminobutyric acid (GABA).

2. The method of claim 1, wherein the bacterium is selected from Bifidobacterium adolescentis, Lactiplantibacillus plantarum, and Limosilactobacillus reuteri.

3. The method of claim 1, wherein the bacterium is selected from Bifidobacterium adolescentis, Lactiplantibacillus plantarum, Limosilactobacillus reuteri, Bifidobacterium animalis, Bifidobacterium dentium, and Lactococcus lactis.

4. The method of claim 1, wherein the bacterium is selected from Bifidobacterium adolescentis IVS-1, Limosilactobacillus reuteri K9-9, and Lactiplantibacillus plantarum IO-6.

5. The method of claim 2, wherein the bacterium is Limosilactobacillus reuteri K9-9.

6. The method of claim 1, wherein the mental health disorder includes one or more of: mood disorder, anxiety disorder, depression, attention deficit hyperactivity disorder (ADHD), social impairment, or panic disorders.

7. The method of claim 1, wherein the administration of the composition increases GABA within a body of the subject.

8. The method of claim 1, wherein the composition is administered by oral administration.

9. The method of claim 1, wherein the bacterium is provided at a concentration of between about 10³ CFUs to about 10¹⁰ CFUs.

10. The method of claim 1, wherein the composition further comprises a prebiotic comprising galactooligosaccharide (GOS), fructooligosaccharide (FOS), inulin, or a combination thereof.

11. The method of claim 1, wherein the composition is further administered with a therapeutic agent comprising an antidepressant, an anxiolytic, stimulants, non-stimulants, or a combination thereof.

12. The method of 11, wherein the therapeutic agent comprises a benzodiazepine, selective serotonin reuptake inhibitors (SSRIs), serotonin and norepinephrine reuptake inhibitors (SNRIs), tricyclic antidepressants (TCAs), monoamine oxidase inhibitors (MAOIs), methylphenidate, amphetamine, atomoxetine, viloxazine, guanfacine, clonidine, or a combination thereof.

13. The method of claim 1, wherein the administration of the composition decreases a responsiveness of a nerve cell.

14. A method of treating a disorder in a subject in need thereof, the method comprising:
administering to the subject a probiotic comprising a therapeutically effective amount of a bacterium capable of producing γ-aminobutyric acid (GABA);
wherein the probiotic is administered at least one time.

15. The method of claim 14, wherein the health disorders include one or more of: chronic pain, fatigue, blood pressure dysregulation, cardiovascular dysfunction, epilepsy, or inflammation.

16. The method of claim 14, wherein the bacterium is selected from Bifidobacterium adolescentis, Lactiplantibacillus plantarum, Limosilactobacillus reuteri, Bifidobacterium animalis, Bifidobacterium dentium, and Lactococcus lactis.

17. The method of claim 14, wherein the bacterium is Bifidobacterium adolescentis IVS-1.

18. The method of claim 14, wherein the bacterium is Limosilactobacillus reuteri K9-9.

19. The method of claim 14, wherein the at least one time comprises one time daily.

20. A method of treating a mental health disorder in a subject in need thereof, the method comprising:
administering to the subject a composition comprising a therapeutically effective amount of a bacterium capable of producing γ-aminobutyric acid (GABA);
wherein the composition comprises a probiotic, the probiotic provided at a concentration of between about 10³ CFUs to about 10¹⁰ CFUs; and
wherein the bacterium is selected from Bifidobacterium adolescentis, Lactiplantibacillus plantarum, and Limosilactobacillus reuteri.


SEQUENCE LISTING (16 sequences)

SEQ ID NO: 1 - DNA, 1525 bp, Bifidobacterium adolescentis
  source 1..1525: mol_type = genomic DNA
tgtggagggttcgattctggctcaggatgaacgctggcggcgtgcttaacacatgcaagtcgaacgggatcccaggagcttgctcctgggtgagagtggcgaacgggtgagtaatgcgtgaccgacctgccccatacaccggaatagctcctggaaacgggtggtaatgccggatgctccagttgaccgcatggtcctctgggaaagcttttgcggtatgggatggggtcgcgtcctatcagcttgatggcggggtaacggcccaccatggcttcgacgggtagccggcctgagagggcgaccggccacattgggactgagatacggcccagactcctacgggaggcagcagtggggaatattgcacaatgggcgcaagcctgatgcagcgacgccgcgtgcgggatgacggccttcgggttgtaaaccgcttttgactgggagcaagcccttcggggtgagtgtacctttcgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgtcactgacgctgaggagcgaaagcgtggggagcgaacaggattagataccctggtagtccacgccgtaaacggtggatgctggatgtggggaccattccacggtctccgtgtcggagccaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacagccgtagagatacggtctcccttcggggcgggttcacaggtggtgcatggtcgtcgtcagctcgtgtcgtgagatgttgggttaagtcccgcaacgagcgcaaccctcgccctgtgttgccagcacgtcgtggtgggaactcacgggggaccgccggggtcaactcggaggaaggtggggatgacgtcagatcatcatgccccttacgtccagggcttcacgcatgctacaatggccggtacaacgggatgcgacactgtgaggtggagcggatcccttaaaaccggtctcagttcggattggagtctgcaacccgactccatgaaggcggagtcgctagtaatcgcggatcagcaacgccgcggtgaatgcgttcccgggccttgtacacaccgcccgtcaagtcatgaaagtgggtagcacccgaagccggtggcccaacctttttggggggagccgtctaaggtgagactcgtgattgggactaagtcgtaacaaggtagccgtaccggaaggtgcggctggatcacctccttt

SEQ ID NO: 2 - DNA, 1563 bp, unidentified
  source 1..1563: mol_type = genomic DNA; note = Lactiplantibacillus plantarum
tttgagagtttgatcctggctcaggacgaacgctggcggcgtgcctaatacatgcaagtcgaacgaactctggtattgattggtgcttgcatcatgatttacatttgagtgagtggcgaactggtgagtaacacgtgggaaacctgcccagaagcgggggataacacctggaaacagatgctaataccgcataacaacttggaccgcatggtccgagcttgaaagatggcttcggctatcacttttggatggtcccgcggcgtattagctagatggtggggtaacggctcaccatggcaatgatacgtagccgacctgagagggtaatcggccacattgggactgagacacggcccaaactcctacgggaggcagcagtagggaatcttccacaatggacgaaagtctgatggagcaacgccgcgtgagtgaagaagggtttcggctcgtaaaactctgttgttaaagaagaacatatctgagagtaactgttcaggtattgacggtatttaaccagaaagccacggctaactacgtgccagcagccgcggtaatacgtaggtggcaagcgttgtccggatttattgggcgtaaagcgagcgcaggcggttttttaagtctgatgtgaaagccttcggctcaaccgaagaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagtatgggtagcaaacaggattagataccctggtagtccataccgtaaacgatgaatgctaagtgttggagggtttccgcccttcagtgctgcagctaacgcattaagcattccgcctggggagtacggccgcaaggctgaaactcaaaggaattgacgggggcccgcacaagcggtggagcatgtggtttaattcgaagctacgcgaagaaccttaccaggtcttgacatactatgcaaatctaagagattagacgttcccttcggggacatggatacaggtggtgcatggttgtcgtcagctcgtgtcgtgagatgttgggttaagtcccgcaacgagcgcaacccttattatcagttgccagcattaagttgggcactctggtgagactgccggtgacaaaccggaggaaggtggggatgacgtcaaatcatcatgccccttatgacctgggctacacacgtgctacaatggatggtacaacgagttgcgaactcgcgagagtaagctaatctcttaaagccattctcagttcggattgtaggctgcaactcgcctacatgaagtcggaatcgctagtaatcgcggatcagcatgccgcggtgaatacgttcccgggccttgtacacaccgcccgtcacaccatgagagtttgtaacacccaaagtcggtggggtaaccttttaggaaccagccgcctaaggtgggacagatgattagggtgaagtcgtaacaaggtagccgtaggagaacctgcggctggatcacctcctt

SEQ ID NO: 3 - DNA, 1573 bp, unidentified
  source 1..1573: mol_type = genomic DNA; note = Limosilactobacillus reuteri
tatgagagtttgatcctggctcaggatgaacgccggcggtgtgcctaatacatgcaagtcgtacgcactggcccaactgattgatggtgcttgcacctgattgacgatggatcaccagtgagtggcggacgggtgagtaacacgtaggtaacctgccccggagcgggggataacatttggaaacagatgctaataccgcataacaacaaaagccacatggcttttgtttgaaagatggctttggctatcactctgggatggacctgcggtgcattagctagttggtaaggtaacggcttaccaaggcgatgatgcatagccgagttgagagactgatcggccacaatggaactgagacacggtccatactcctacgggaggcagcagtagggaatcttccacaatgggcgcaagcctgatggagcaacaccgcgtgagtgaagaagggtttcggctcgtaaagctctgttgttggagaagaacgtgcgtgagagtaactgttcacgcagtgacggtatccaaccagaaagtcacggctaactacgtgccagcagccgcggtaatacgtaggtggcaagcgttatccggatttattgggcgtaaagcgagcgcaggcggttgcttaggtctgatgtgaaagccttcggcttaaccgaagaagtgcatcggaaaccgggcgacttgagtgcagaagaggacagtggaactccatgtgtagcggtggaatgcgtagatatatggaagaacaccagtggcgaaggcggctgtctggtctgcaactgacgctgaggctcgaaagcatgggtagcgaacaggattagataccctggtagtccatgccgtaaacgatgagtgctaggtgttggagggtttccgcccttcagtgccggagctaacgcattaagcactccgcctggggagtacgaccgcaaggttgaaactcaaaggaattgacgggggcccgcacaagcggtggagcatgtggtttaattcgaagctacgcgaagaaccttaccaggtcttgacatcttgcgctaaccttagagataaggcgttcccttcggggacgcaatgacaggtggtgcatggtcgtcgtcagctcgtgtcgtgagatgttgggttaagtcccgcaacgagcgcaacccttgttactagttgccagcattaagttgggcactctagtgagactgccggtgacaaaccggaggaaggtggggacgacgtcagatcatcatgccccttatgacctgggctacacacgtgctacaatggacggtacaacgagtcgcaagctcgcgagagtaagctaatctcttaaagccgttctcagttcggactgtaggctgcaactcgcctacacgaagtcggaatcgctagtaatcgcggatcagcatgccgcggtgaatacgttcccgggccttgtacacaccgcccgtcacaccatgggagtttgtaacgcccaaagtcggtggcctaacctttatggagggagccgcctaaggcgggacagatgactggggtgaagtcgtaacaaggtagccgtaggagaacctgcggctggatcacctccttt

SEQ ID NO: 4 - DNA, 1433 bp, Bifidobacterium adolescentis
  source 1..1433: mol_type = genomic DNA
tgtggagggttcgattctggctcaggatgaacgctggcggcgtgcttaacacatgcaagtcgaacgggatcccaggagcttgctcctgggtgagagtggcgaacgggtgagtaatgcgtgaccgacctgccccatacaccggaatagctcctggaaacgggtggtaatgccggatgctccagttggatgcatgtccttctgggaaagattcatcggtatgggatggggtcgcgtcctatcagcttgatggcggggtaacggcccaccatggcttcgacgggtagccggcctgagagggcgaccggccacattgggactgagatacggcccagactcctacgggaggcagcagtggggaatattgcacaatgggcgcaagcctgatgcagcgacgccgcgtgcgggatgacggccttcgggttgtaaaccgcttttgactgggagcaagcccttcggggtgagtgtacctttcgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgtcactgacgctgaggagcgaaagcgtggggagcgaacaggattagataccctggtagtccacgccgtaaacggtggatgctggatgtggggaccattccacggtctccgtgtcggagccaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacagccccagagatggggcctcccttcggggcgggttcacaggtggtgcatggtcgtcgtcagctcgtgtcgtgagatgttgggttaagtcccgcaacgagcgcaaccctcgccctgtgttgccagcacgtcgtggtgggaactcacgggggaccgccggggtcaactcggaggaaggtggggatgacgtcagatcatcatgccccttacgtccagggcttcacgcatgctacaatggccggtacaacgggatgcgacaccgcgaggtggagcggatcccttaaaaccggtctcagttcggattggagtctgcaacccgactccatgaaggcggagtcgctagtaatcgcggatcagcaacgccgcggtgaatgcgttcccgggccttgtacacaccgcccgtcaagtcatgaaagtgggtagcacccgaagccggtggcccaacctt

SEQ ID NO: 5 - DNA, 1524 bp, Bifidobacterium adolescentis
  source 1..1524: mol_type = genomic DNA
tgtggagggttcgattctggctcaggatgaacgctggcggcgtgcttaacacatgcaagtcgaacgggatcccaggagcttgctcctgggtgagagtggcgaacgggtgagtaatgcgtgaccgacctgccccatacaccggaatagctcctggaaacgggtggtaatgccggatgctccagttggatgcatgtccttctgggaaagattcatcggtatgggatggggtcgcgtcctatcagcttgatggcggggtaacggcccaccatggcttcgacgggtagccggcctgagagggcgaccggccacattgggactgagatacggcccagactcctacgggaggcagcagtggggaatattgcacaatgggcgcaagcctgatgcagcgacgccgcgtgcgggatgacggccttcgggttgtaaaccgcttttgactgggagcaagcccttcggggtgagtgtacctttcgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgtcactgacgctgaggagcgaaagcgtggggagcgaacaggattagataccctggtagtccacgccgtaaacggtggatgctggatgtggggaccattccacggtctccgtgtcggagccaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacagccgtagagatacggtctcccttcggggcgggttcacaggtggtgcatggtcgtcgtcagctcgtgtcgtgagatgttgggttaagtcccgcaacgagcgcaaccctcgccctgtgttgccagcacgtcgtggtgggaactcacgggggaccgccggggtcaactcggaggaaggtggggatgacgtcagatcatcatgccccttacgtccagggcttcacgcatgctacaatggccggtacaacgggatgcgacctcgtgagggggagcggatcccttaaaaccggtctcagttcggattggagtctgcaacccgactccatgaaggcggagtcgctagtaatcgcggatcagcaacgccgcggtgaatgcgttcccgggccttgtacacaccgcccgtcaagtcatgaaagtgggtagcacccgaagccggtggcccaaccttttggggggagccgtctaaggtgagactcgtgattgggactaagtcgtaacaaggtagccgtaccggaaggtgcggctggatcacctccttt

SEQ ID NO: 6 - DNA, 1431 bp, Bifidobacterium adolescentis
  source 1..1431: mol_type = genomic DNA
gagtggcgaacgggtgagtaatgcgtgaccgacctgccccatacaccggaatagctcctggaaacgggtggtaatgccggatgctccagttggatgcatgtccttctgggaaagattcatcggtatgggatggggtcgcgtcctatcagcttgatggcggggtaacggcccaccatggcttcgacgggtagccggcctgagagggcgaccggccacattgggactgagatacggcccagactcctacgggaggcagcagtggggaatattgcacaatgggcgcaagcctgatgcagcgacgccgcgtgcgggatgacggccttcgggttgtaaaccgcttttgactgggagcaagcccttcggggtgagtgtacctttcgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgtcactgacgctgaggagcgaaagcgtggggagcgaacaggattagataccctggtagtccacgccgtaaacggtggatgctggatgtggggaccattccacggtctccgtgtcggagccaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacagccgtagagatacggtctcccttcggggcgggttcacaggtggtgcatggtcgtcgtcagctcgtgtcgtgagatgttgggttaagtcccgcaacgagcgcaaccctcgccctgtgttgccagcacgtcgtggtgggaactcacgggggaccgccggggtcaactcggaggaaggtggggatgacgtcagatcatcatgccccttacgtccagggcttcacgcatgctacaatggccggtacaacgggatgcgacctcgtgagggggagcggatcccttaaaaccggtctcagttcggattggagtctgcaacccgactccatgaaggcggagtcgctagtaatcgcggatcagcaacgccgcggtgaatgcgttcccgggccttgtacacaccgcccgtcaagtcatgaaagtgggtagcacccgaagccggtggcccaaccttttggggggagccgtctaaggtgagactcgtgattgggactaagtcgtaacaaggtagccgtaccggaaggtgcggctggatcacctccttt

SEQ ID NO: 7 - DNA, 783 bp, Bifidobacterium adolescentis
  source 1..783: mol_type = genomic DNA
gtatgggatggggtcgcgtcctatcagcttgatggcggggtaacggcccaccatggcttcgacgggtagccggcctgagagggcgaccggccacattgggactgagatacggcccagactcctacgggaggcagcagtggggaatattgcacaatgggcgcaagcctgatgcagcgacgccgcgtgcgggatgacggccttcgggttgtaaaccgcttttgactgggagcaagcccttcggggtgagtgtacctttcgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgtcactgacgctgaggagcgaaagcgtggggagcgaacaggattagataccctggtagtccacgccgtaaacggtggatgctggatgtggggaccattccacggtctccgtgtcggagccaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacagccccagaga

SEQ ID NO: 8 - DNA, 1524 bp, Bifidobacterium adolescentis
  source 1..1524: mol_type = genomic DNA
tgtggagggttcgattctggctcaggatgaacgctggcggcgtgcttaacacatgcaagtcgaacgggatcccaggagcttgctcctgggtgagagtggcgaacgggtgagtaatgcgtgaccgacctgccccatacaccggaatagctcctggaaacgggtggtaatgccggatgctccagttggatgcatgtccttctgggaaagattcatcggtatgggatggggtcgcgtcctatcagcttgatggcggggtaacggcccaccatggcttcgacgggtagccggcctgagagggcgaccggccacattgggactgagatacggcccagactcctacgggaggcagcagtggggaatattgcacaatgggcgcaagcctgatgcagcgacgccgcgtgcgggatgacggccttcgggttgtaaaccgcttttgactgggagcaagcccttcggggtgagtgtacctttcgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgtcactgacgctgaggagcgaaagcgtggggagcgaacaggattagataccctggtagtccacgccgtaaacggtggatgctggatgtggggaccattccacggtctccgtgtcggagccaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacagccgtagagatacggtctcccttcggggcgggttcacaggtggtgcatggtcgtcgtcagctcgtgtcgtgagatgttgggttaagtcccgcaacgagcgcaaccctcgccctgtgttgccagcacgtcgtggtgggaactcacgggggaccgccggggtcaactcggaggaaggtggggatgacgtcagatcatcatgccccttacgtccagggcttcacgcatgctacaatggccggtacaacgggatgcgacactgtgaggtggagcggatcccttaaaaccggtctcagttcggattggagtctgcaacccgactccatgaaggcggagtcgctagtaatcgcggatcagcaacgccgcggtgaatgcgttcccgggccttgtacacaccgcccgtcaagtcatgaaagtgggtagcacccgaagccggtggcccaaccttttggggggagccgtctaaggtgagactcgtgattgggactaagtcgtaacaaggtagccgtaccggaaggtgcggctggatcacctccttt

SEQ ID NO: 9 - DNA, 1309 bp, Bifidobacterium adolescentis
  source 1..1309: mol_type = genomic DNA
gtatgggatggggtcgcgtcctatcagcttgatggcggggtaacggcccaccatggcttcgacgggtagccggcctgagagggcgaccggccacattgggactgagatacggcccagactcctacgggaggcagcagtggggaatattgcacaatgggcgcaagcctgatgcagcgacgccgcgtgcgggatgacggccttcgggttgtaaaccgcttttgactgggagcaagcccttcggggtgagtgtacctttcgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgtcactgacgctgaggagcgaaagcgtggggagcgaacaggattagataccctggtagtccacgccgtaaacggtggatgctggatgtggggaccattccacggtctccgtgtcggagccaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacagccccagagatggggcctcccttcggggcgggttcacaggtggtgcatggtcgtcgtcagctcgtgtcgtgagatgttgggttaagtcccgcaacgagcgcaaccctcgccctgtgttgccagcacgtcgtggtgggaactcacgggggaccgccggggtcaactcggaggaaggtggggatgacgtcagatcatcatgccccttacgtccagggcttcacgcatgctacaatggccggtacaacgggatgcgacaccgcgaggtggagcggatcccttaaaaccggtctcagttcggattggagtctgcaacccgactccatgaaggcggagtcgctagtaatcgcggatcagcaacgccgcggtgaatgcgttcccgggccttgtacacaccgcccgtcaagtcatgaaagtgggtagcacccgaagccggtggcccaaccttttggggggagccgtctaaggtgagactcgtgattgggactaagtcgtaacaaggtagccgtaccggaaggtgcggctggatcacctccttt

SEQ ID NO: 10 - DNA, 899 bp, Bifidobacterium adolescentis
  source 1..899: mol_type = genomic DNA
gtatgggatggggtcgcgtcctatcagcttgatggcggggtaacggcccaccatggcttcgacgggtagccggcctgagagggcgaccggccacattgggactgagatacggcccagactcctacgggaggcagcagtggggaatattgcacaatgggcgcaagcctgatgcagcgacgccgcgtgcgggatgacggccttcgggttgtaaaccgcttttgactgggagcaagcccttcggggtgagtgtacctttcgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgtcactgacgctgaggagcgaaagcgtggggagcgaacaggattagataccctggtagtccacgccgtaaacggtggatgctggatgtggggaccattccacggtctccgtgtcggagccaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacagccgtagagatacggtctcccttcggggcgggttcacaggtggtgcatggtcgtcgtcagctcgtgtcgtgagatgttgggttaagtcccgcaacgagcgcaaccctcgccctgtgttgccagcac

SEQ ID NO: 11 - DNA, 1524 bp, Bifidobacterium adolescentis; identical to SEQ ID NO: 5
  source 1..1524: mol_type = genomic DNA

SEQ ID NO: 12 - DNA, 1528 bp, Bifidobacterium dentium
  source 1..1528: mol_type = genomic DNA
tatggagggttcgattctggctcaggatgaacgctggcggcgtgcttaacacatgcaagtcgaacgggatcccgggggttcgcttccgggtgagagtggcgaacgggtgagtaatgcgtgaccgacctgccccatacaccggaatagctcctggaaacgggtggtaatgccggatgctccggttggatgcatgtccttccgggaaagattccatcggtatgggatggggtcgcgtcctatcagcttgatggcggggtaacggcccaccatggcttcgacgggtagccggcctgagagggcgaccggccacattgggactgagatacggcccagactcctacgggaggcagcagtggggaatattgcacaatgggcgcaagcctgatgcagcgacgccgcgtgcgggatggaggccttcgggttgtaaaccgcttttgatcgggagcaagcccttcggggtgagtgtacctttcgaataagcaccggctaactacgtgccagcagccgcggtaatacgtagggtgcaagcgttatccggaattattgggcgtaaagggctcgtaggcggttcgtcgcgtccggtgtgaaagcccatcgcttaacggtgggtctgcgccgggtacgggcgggctggagtgcggtaggggagactggaattcccggtgtaacggtggaatgtgtagatatcgggaagaacaccaatggcgaaggcaggtctctgggccgtcactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacggccgtagagatatggcctcccttcggggcgggttcacaggtggtgcatggtcgtcgtcagctcgtgtcgtgagatgttgggttaagtcccgcaacgagcgcaaccctcgccctgtgttgccagcacgtcatggtgggaactcacgggggaccgccggggtcaactcggaggaaggtggggatgacgtcagatcatcatgccccttacgtccagggcttcacgcatgctacaatggccggtacagcgggatgcgacatggcgacatggagcggatccctgaaaaccggtctcagttcggattggagtctgcaacccgactccatgaaggcggagtcgctagtaatcgcggatcagcaacgccgcggtgaatgcgttcccgggccttgtacacaccgcccgtcaagtcatgaaagtgggcagcacccgaagccggtggcctaaccccttgtgggatggagccgtctaaggtgaggctcgtgattgggactaagtcgtaacaaggtagccgtaccggaaggtgcggctggatcacctccttt

SEQ ID NO: 13 - DNA, 1327 bp, unidentified
  source 1..1327: mol_type = genomic DNA; note = Lactiplantibacillus plantarum
tatcacttttggatggtcccgcggcgtattagctagatggtggggtaacggctcaccatggcaatgatacgtagccgacctgagagggtaatcggccacattgggactgagacacggcccaaactcctacgggaggcagcagtagggaatcttccacaatggacgaaagtctgatggagcaacgccgcgtgagtgaagaagggtttcggctcgtaaaactctgttgttaaagaagaacatatctgagagtaactgttcaggtattgacggtatttaaccagaaagccacggctaactacgtgccagcagccgcggtaatacgtaggtggcaagcgttgtccggatttattgggcgtaaagcgagcgcaggcggttttttaagtctgatgtgaaagccttcggctcaaccgaagaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagtatgggtagcaaacaggattagataccctggtagtccataccgtaaacgatgaatgctaagtgttggagggtttccgcccttcagtgctgcagctaacgcattaagcattccgcctggggagtacggccgcaaggctgaaactcaaaggaattgacgggggcccgcacaagcggtggagcatgtggtttaattcgaagctacgcgaagaaccttaccaggtcttgacatactatgcaaatctaagagattagacgttcccttcggggacatggatacaggtggtgcatggttgtcgtcagctcgtgtcgtgagatgttgggttaagtcccgcaacgagcgcaacccttattatcagttgccagcattaagttgggcactctggtgagactgccggtgacaaaccggaggaaggtggggatgacgtcaaatcatcatgccccttatgacctgggctacacacgtgctacaatggatggtacaacgagttgcgaactcgcgagagtaagctaatctcttaaagccattctcagttcggattgtaggctgcaactcgcctacatgaagtcggaatcgctagtaatcgcggatcagcatgccgcggtgaatacgttcccgggccttgtacacaccgcccgtcacaccatgagagtttgtaacacccaaagtcggtggggtaaccttttaggaaccagccgcctaaggtgggacagatgattagggtgaagtcgtaacaaggtagccgtaggagaacctgcggctggatcacctcctt

SEQ ID NO: 14 - DNA, 1563 bp, unidentified
  source 1..1563: mol_type = genomic DNA; note = Lactiplantibacillus plantarum
tttgagagtttgatcctggctcaggacgaacgctggcggcgtgcctaatacatgcaagtcgaacgaactctggtattgattggtgcttgcatcatgatttacatttgagtgagtggcgaactggtgagtaacacgtgggaaacctgcccagaagcgggggataacacctggaaacagatgctaataccgcataacaacttggaccgcatggtccgagtttgaaagatggcttcggctatcacttttggatggtcccgcggcgtattagctagatggtggggtaacggctcaccatggcaatgatacgtagccgacctgagagggtaatcggccacattgggactgagacacggcccaaactcctacgggaggcagcagtagggaatcttccacaatggacgaaagtctgatggagcaacgccgcgtgagtgaagaagggtttcggctcgtaaaactctgttgttaaagaagaacatatctgagagtaactgttcaggtattgacggtatttaaccagaaagccacggctaactacgtgccagcagccgcggtaatacgtaggtggcaagcgttgtccggatttattgggcgtaaagcgagcgcaggcggttttttaagtctgatgtgaaagccttcggctcaaccgaagaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagtatgggtagcaaacaggattagataccctggtagtccataccgtaaacgatgaatgctaagtgttggagggtttccgcccttcagtgctgcagctaacgcattaagcattccgcctggggagtacggccgcaaggctgaaactcaaaggaattgacgggggcccgcacaagcggtggagcatgtggtttaattcgaagctacgcgaagaaccttaccaggtcttgacatactatgcaaatctaagagattagacgttcccttcggggacatggatacaggtggtgcatggttgtcgtcagctcgtgtcgtgagatgttgggttaagtcccgcaacgagcgcaacccttattatcagttgccagcattaagttgggcactctggtgagactgccggtgacaaaccggaggaaggtggggatgacgtcaaatcatcatgccccttatgacctgggctacacacgtgctacaatggatggtacaacgagttgcgaactcgcgagagtaagctaatctcttaaagccattctcagttcggattgtaggctgcaactcgcctacatgaagtcggaatcgctagtaatcgcggatcagcatgccgcggtgaatacgttcccgggccttgtacacaccgcccgtcacaccatgagagtttgtaacacccaaagtcggtggggtaaccttttaggaaccagccgcctaaggtgggacagatgattagggtgaagtcgtaacaaggtagccgtaggagaacctgcggctggatcacctcctt

SEQ ID NO: 15 - DNA, 1563 bp, unidentified; identical to SEQ ID NO: 14
  source 1..1563: mol_type = genomic DNA; note = Lactiplantibacillus plantarum

SEQ ID NO: 16 - DNA, 1573 bp, unidentified
  source 1..1573: mol_type = genomic DNA; note = Limosilactobacillus reuteri
tatgagagtttgatcctggctcaggatgaacgccggcggtgtgcctaatacatgcaagtcgtacgcactggcccaactgattgatggtgcttgcacctgattgacgatggatcaccagtgagtggcggacgggtgagtaacacgtaggtaacctgccccggagcgggggataacatttggaaacagatgctaataccgcataacaacaaaagccacatggcttttgtttgaaagatggctttggctatcactctgggatggacctgcggtgcattagctagttggtaaggtaacggcttaccaaggcgatgatgcatagccgagttgagagactgatcggccacaatggaactgagacacggtccatactcctacgggaggcagcagtagggaatcttccacaatgggcgcaagcctgatggagcaacaccgcgtgagtgaagaagggtttcggctcgtaaagctctgttgttggagaagaacgtgcgtgagagtaactgttcacgcagtgacggtatccaaccagaaagtcacggctaactacgtgccagcagccgcggtaatacgtaggtggcaagcgttatccggatttattgggcgtaaagcgagcgcaggcggttgcttaggtctgatgtgaaagccttcggcttaaccgaagaagtgcatcggaaaccgggcgacttgagtgcagaagaggacagtggaactccatgtgtagcggtggaatgcgtagatatatggaagaacaccagtggcgaaggcggctgtctggtctgcaactgacgctgaggctcgaaagcatgggtagcgaacaggattagataccctggtagtccatgccgtaaacgatgagtgctaggtgttggagggtttccgcccttcagtgccggagctaacgcattaagcactccgcctggggagtacgaccgcaaggttgaaactcaaaggaattgacgggggcccgcacaagcggtggagcatgtggtttaattcgaagctacgcgaagaaccttaccaggtcttgacatcttgcgctaaccttagagataaggcgttcccttcggggacgcaatgacaggtggtgcatggtcgtcgtcagctcgtgtcgtgagatgttgggttaagtcccgcaacgagcgcaacccttgttactagttgccagcattaagttgggcactctagtgagactgccggtgacaaaccggaggaaggtggggacgacgtcagatcatcatgccccttatgacctgggctacacacgtgctacaatggacggtacaacgagtcgcaaactcgcgagagtaagctaatctcttaaagccgttctcagttcggactgtaggctgcaactcgcctacacgaagtcggaatcgctagtaatcgcggatcagcatgccgcggtgaatacgttcccgggccttgtacacaccgcccgtcacaccatgggagtttgtaacgcccaaagtcggtggcctaacctttatggagggagccgcctaaggcgggacagatgactggggtgaagtcgtaacaaggtagccgtaggagaacctgcggctggatcacctccttt